(19) United States
(12) Patent Application Publication
(10) Pub. No.: US 2016/0136175 A1
(43) Pub. Date: May 19, 2016

(54) COMBINATION OF A PHOSPHATIDYLINOSITOL-3-KINASE (PI3K) INHIBITOR AND A MTOR INHIBITOR

(71) Applicants: Carlos Garcia-Echeverria, Basel (CH); Sauveur-Michel Maira, Habsheim (FR)

(72) Inventors: Carlos Garcia-Echeverria, Basel (CH); Sauveur-Michel Maira, Habsheim (FR)

(73) Assignee: Novartis AG, Basel (CH)

(21) Appl. No.: 14/950,869

(22) Filed: Nov. 24, 2015

Related U.S. Application Data
(63) Continuation of application No. 13/123,956, filed on Apr. 13, 2011.

(30) Foreign Application Priority Data
Oct. 31, 2008 (EP) 08168044.9

(51) Int. Cl. A61K 31/5377; A61K 31/436

(52) U.S. Cl. CPC A61K 31/5377; A61K 31/436

(57) ABSTRACT

The invention relates to a pharmaceutical combination which comprises (a) a phosphoinositide 3-kinase inhibitor compound of formula (I) and (b) a mTOR inhibitor for the treatment of a target of rapamycin (mTOR) kinase dependent disease, especially a cancer disease; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

### FIELD OF THE INVENTION

The present invention relates to a pharmaceutical combination comprising a phosphalidylinositol-3-Kinase (PI3K) inhibitor compound which is a pyrimidine derivative and a mTOR inhibitor, and the uses of such a combination in the treatment proliferative diseases, more specifically of mammalian target of rapamycin (mTOR) kinase dependent diseases.

### BACKGROUND OF THE INVENTION

It has been shown that mTOR inhibition can induce upstream insulin-like growth factor 1 receptor (IGF-1R) signaling resulting in AKT activation in cancer cells. This phenomenon has been suggested to play a role in the attenuation of cellular responses to mTOR inhibition and may attenuate the clinical activity of mTOR inhibitors. Increase in pAKT has for instance been found in approximately 50% in the tumours of all patients in a Phase I study in patients with advanced solid tumours (Taberno et al., Journal of Clinical Oncology, 26 (2008), pp 1603-1610). In spite of numerous treatment options for proliferative disease patients, there remains a need for effective and safe therapeutic agents and a need for their preferential use in combination therapy.

### SUMMARY OF INVENTION

It has been now been found in accordance with the present invention that a PI3K inhibitor reduces or blocks the phosphorylation and activation of AKT by mTOR inhibitors. Accordingly, the present invention provides a method to reduce or block the phosphorylation and activation of AKT by mTOR inhibitors comprising administering a PI3K inhibitor to a warm-blooded animal in need thereof.

In another embodiment, the present invention provides a method of treating a proliferative disease dependent on acquired phosphorylation and activation of AKT during treatment with an mTOR inhibitor comprising administering a therapeutically effective amount of a PI3K inhibitor to a warm-blooded animal in need thereof.

In another embodiment, the present invention relates to a method of treating a proliferative disease which has become resistant or has a decreased sensitivity to the treatment with an mTOR inhibitor comprising administering a therapeutically effective amount of a PI3K inhibitor to a warm-blooded animal in need thereof. The resistance is e.g. due to posphorylation and activation of AKT.

In a further aspect the present invention provides a method for improving efficacy of the treatment of a proliferative disease with an mTOR inhibitor comprising administering a combination comprising a PI3K inhibitor and a mTOR inhibitor to a warm-blooded animal in need thereof.

In one aspect the present invention provides a pharmaceutical composition comprising a PI3K inhibitor compound and at least one mTOR inhibitor.

In another aspect the present invention provides the use of a PI3K inhibitor compound and at least one mTOR inhibitor for the manufacture of a medicament for the treatment or prevention of a proliferative disease.

In another aspect the present invention provides a method of treating or preventing a proliferative by administering a PI3K inhibitor compound and at least one mTOR inhibitor.

In another aspect the present invention provides pharmaceutical combination comprising a PI3K inhibitor compound and at least one mTOR inhibitor for use in treating or preventing a proliferative disease.

In another aspect the present invention provides a combination of a PI3K inhibitor compound and an mTOR inhibitor selected from the group consisting of RAD rapamycin (sirolimus) and denvatlves/analogs thereof such as everolimus or RAD001, CCI-779, ABT578. SAR543, ascomycin (an ethyl analog of FK506), AP23573, AP23841, KU-0063794, INK-128, EX2044, EX3855, EX7518, AZD08055 and OSI027, wherein the active ingredients are present in each case in free form or in the form of a pharmaceutically acceptable salt, and optionally at least one pharmaceutically acceptable carrier, for simultaneous, separate or sequential use for the treatment of mammalian target of rapamycin (mTOR) kinase dependent diseases.
In another aspect the PI3K inhibitor is 5-(2,6-di-morpholin-4-yl-pyrimidin-4-yl)-4-trifluoromethyl-pyridin-2-ylamine (Compound I).

### DETAILED DESCRIPTION OF THE FIGURES

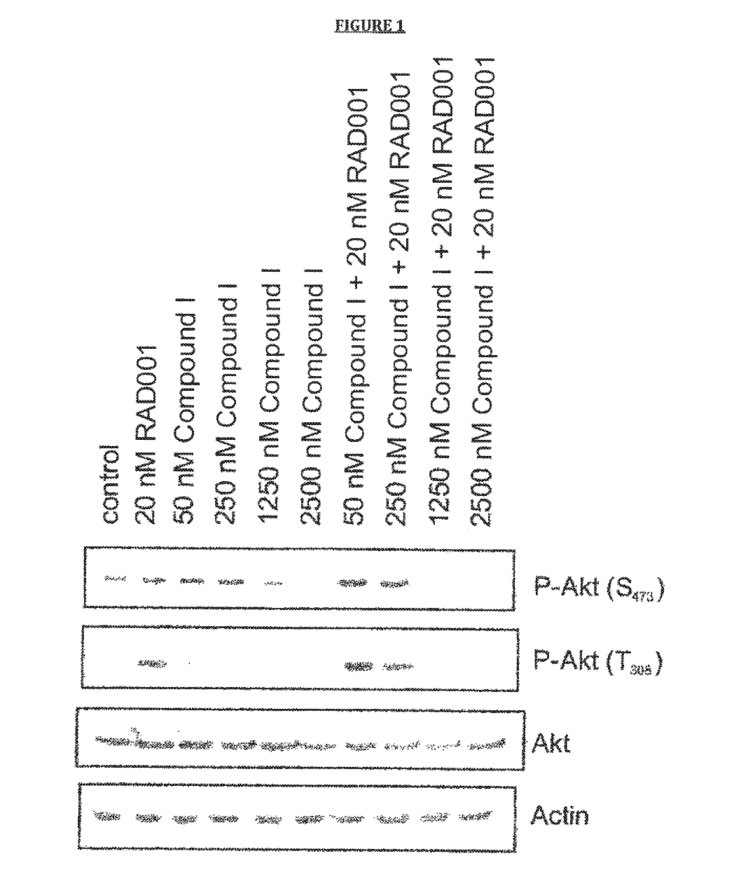

FIG. 1 shows the AKT phosphorylation levels in presence of everolimus (RAD001) and everolimus (RAD001) in combination with Compound I in BT474 breast tumor cells.

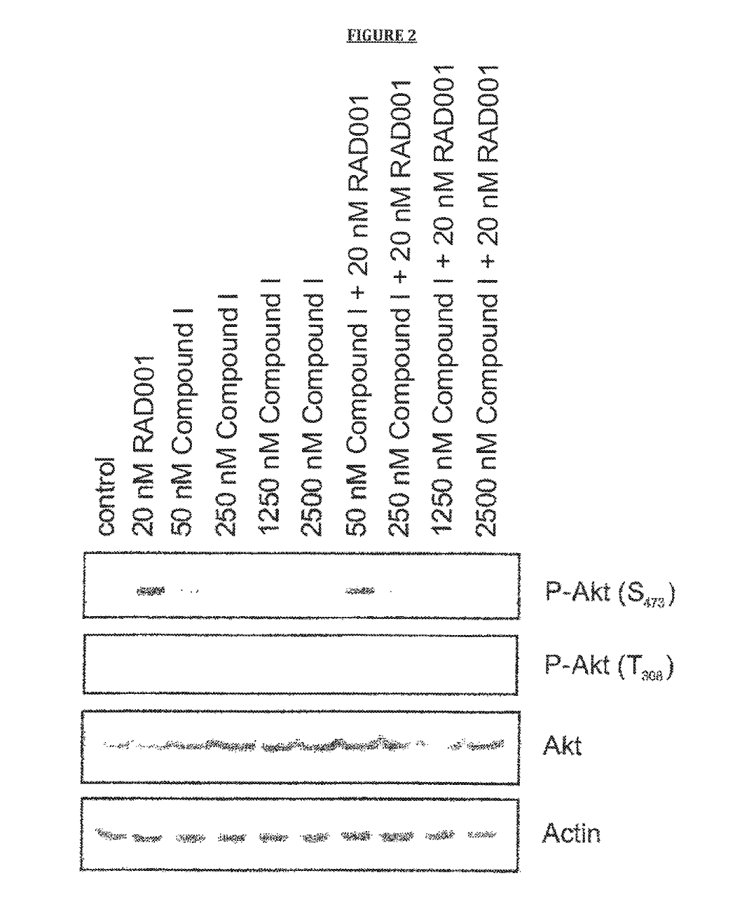

FIG. 2 shows the AKT phosphorylation levels in presence of everolimus (RAD001) and everolimus (RAD001) in combination with Compound I in MDA-MB-231 breast tumor cells.

### DETAILED DESCRIPTION OF THE INVENTION

WO07/084786 describes pyrimidine derivatives, which have been found the activity of lipid kinases, such as PI3-kinases. Specific pyrimidine derivatives which are suitable for the present invention, their preparation and suitable pharmaceutical formulations containing the same are described in WO07/084786 and include compounds of formula (I)

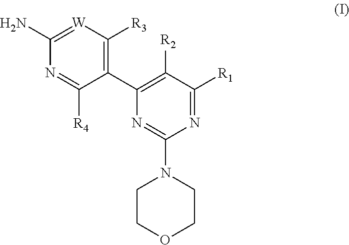

or a stereoisomer, tautomer, or pharmaceutically acceptable salt thereof, wherein,
W is CRw or N, wherein Rw is selected from the group consisting of
(1) hydrogen,
(2) cyano,
(3) halogen,
(4) methyl,
(5) trifluoromethyl,
(6) sulfonamide:
R₁ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NR₁ₐSO₂R_{1b},
(18) —OCOR₁ₐ,
(19) —OR₁ₐ,
(20) —SR₁ₐ,
(21) —SOR₁ₐ,
(22) —SO₂R₁ₐ, and
(23) —SO₂NR₁ₐR_{1b},
wherein R₁ₐ, and R_{1b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
R₂ is selected from the group consisting
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b},
wherein R₂ₐ, and R_{2b} are independently selected from the group consisting of
(a) hydrogen, and
(b) substituted or unsubstituted alkyl;
R₃ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) unsubstituted and unsubstituted cycloalkyl,
(12) —COR₃ₐ,
(13) —NR₃ₐR_{3b},
(14) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ, and
(20) —SO₂NR₃ₐR_{3b},
wherein R₃ₐ, and R_{3b} are independently selected from the group consisting of
(a) hydrogen,
(b) substituted of unsubstituted alkyl,
(c) substituted and unsubstituted aryl,
(d) substituted and unsubstituted heteroaryl,
(e) substituted and unsubstituted heterocycyl, and
(f) substituted and unsubstituted cycloalkyl; and
R₄ is selected from the group consisting of
(1) hydrogen, and
(2) halogen.

The radicals and symbols as used in the definition of a compound of formula (I) have the meanings as disclosed in WO07/084786 which publication is hereby incorporated into the present application by reference.

A preferred compound of the present invention is a compound which is specifically described in WO07/084786. A very preferred compound of the present invention is 5-(2,6-di-morpholin-4-yl-pyrimidin-4-yl)-4-trifluoromethyl-pyridin-2-ylamine (Compound I). The synthesis of 5-(2,6-di-morpholin-yl-pyrimidin-4-yl)-trifluoromethyl-pyridin-2-ylamine is described in WO07/084786 as Example 10.

Combinations of the present invention include compounds which target, decrease or inhibit the activity/function of serine/theronine mTOR kinase. Such compounds will be referred to as “mTOR inhibitors” and Include but is not limited to compounds, proteins or antibodies which target/inhibit members of the mTOR kinase family, e.g., RAD, rapamycin (sirolimus) and derivatives/analogs thereof such as everolimus or RAD001 or compounds that inhibit the kinase activity of mTOR by directly binding to the ATP-binding deft of the enzyme. Sirolimus is also known by the name RAPAMUNE and everolimus or RAD001 by the name CERTICAN. Other compounds, proteins or antibodies which target/Inhibit members of the mTOR kinase family include CCI-779, ABT578, SAR543, and ascomycin which is an ethyl analog of FK506. Also included are AP23573, AP23841, KU-0063794, INK-128, EX2044, EX3855, EX7518, AZD08055 and OSI027. A particularly preferred compound in accordance with the present invention is RAD001.

Suitable mTOR inhibitors include e.g.:
I. Rapamycin which is an immunosuppressive lactam macrolide that is produced by Streptomyces hygroscopicus.
II. Rapamycin derivatives such as:
   a. substituted rapamycin e.g. a 40-O-substituted rapamycin e.g. as described in U.S. Pat. No. 5,258,389. WO 94/09010, WO 92/05179. U.S. Pat. No. 5,118,877. U.S. Pat. No. 5,118,678. U.S. Pat. No. 5,100,883. U.S. Pat. No. 5,151,413. U.S. Pat. No. 5,120,842. WO 93/11130. WO 94/02136, WO 94/02485 and WO 95/14023 all of which are incorporated herein by reference;
   b. a 16-O-substituted rapamycin e.g. as disclosed in WO 94/02136, WO 95/16691 and WO 96/41807, the contents of which are incorporated herein by reference;
   c. a 32-hydrogenated rapamycin e.g. as described in WO 96/41807 and U.S. Pat. No. 5,256,790, incorporated herein by reference.
   d. Preferred rapamycin derivatives are compounds of formula (II)

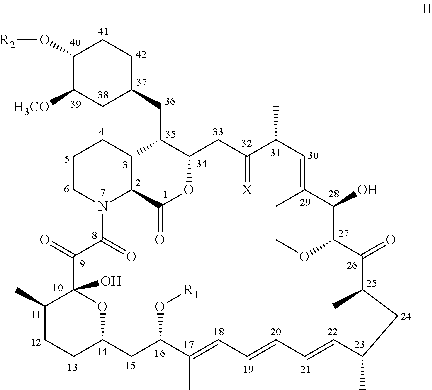

wherein
R₁ is CH₃ or C₃₋₆alkynyl,
R₂ is H or —CH₂—CH₂—OH, 3-hydroxy-2-(hydroxymethyl)-2-methyl-propanoyl or tetrazolyl,
and X is ═O, (H,H) or (H,OH)
provided that R₂ is other than H when X is ═O and R₁ is CH₃,
or a prodrug thereof when R₂ is —CH₂—CH₂—OH, e.g. a physiologically hydrolysable ether thereof.

Compounds of formula (II) are disclosed e.g. in WO 94/09010, WO 95/16691 or WO 96/41807, which are incorporated herein by reference. They may be prepared as disclosed or by analogy to the procedures described in these references

Preferred compounds are 32-deoxorapamycin, 16-pent-2-ynyloxy-32-deoxorapamycin, 16-pent-2-ynyloxy-32(S)dihydro-rapamycin, 16-pent-2-ynyloxy-32(S)-dihydro-40-O-(2-hydroxyethyl)-rapamycin and, more preferably, 40-O-(2-hydroxyethyl)-rapamycin, disclosed as Example 8 in WO 94/09010.

Particularly preferred rapamycin derivatives of formula (II) are 40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 32-deoxorapamycin, 16-pent-2-ynyloxy-32(S)-dihydro rapamycin, or TAFA-93.
e. Rapamycin derivatives also include so-called rapalogs, e.g. as disclosed in WO 98/02441 and WO 01/14387, e.g. AP23573, AP23464, or AP23841.

Rapamycin and derivatives thereof have, on the basis of observed activity, e.g. binding to macrophilin-12 (also known as FK-506 binding protein or FKBP-12), e.g. as described in WO 94/09010. WO 95/16691 or WO 96/41807, been found to be useful e.g. as immuno-suppressant, e.g. in the treatment of acute allograft rejection.
III. Ascomycin, which is an ethyl analog of FK506.
IV. AZD08055 and OSI127, which are compounds that inhibit the kinase activity of mTOR by directly binding to the ATP-binding cleft of the enzyme

Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

In one aspect the present invention provides a pharmaceutical composition comprising a PI3K Inhibitor compound of formula (I) or 5-(2,6-di-morpholin-4-yl-pyrimidin-4-yl)-4-trifluoromethyl-pyridin-2-ylamine as described above and at least one mTOR inhibitor.

In another aspect the present invention provides the use of a PI3K inhibitor compound of formula (I) or 5-(2,6-di-morpholin-4-yl-pyrimidin-4-yl)-4-trifluoromethyl-pyridin-2-ylamine and at least one mTOR inhibitor for the manufacture of a medicament for the treatment or prevention of a proliferative disease.

In a further aspect the present invention provides a compound of formula (I) or 5-(2,6-di-morpholin-4-yl-pyrimidin-4-yl)-4-trifluoromethyl-pyridin-2-ylamine and at least one mTOR inhibitor for use in treating or preventing 3 proliferative disease.

In another aspect the present invention provides a method of treating or preventing a proliferative by administering a compound of formula (I) or 5-(2,6-di-morpholin-4-yl-pyrimidin-4-yl)-4-trifluoromethyl-pyridin-2-ylamine and at least one mTOR inhibitor.

In another aspect the present invention provides pharmaceutical combination comprising a compound of formula (t) or 5-(2,6-di-morpholin-4-yl-pyrimidin-4-yl)-4-trifluoromethyl-pyridin-2-ylamine and at least one mTOR inhibitor for use in treating or preventing a proliferative disease.

In another aspect the present invention provides a combination of a compound of formula (I) 5-(2,6-di-morpholin-4-yl-pyrimidin-4-yl)-4-trifluoromethyl-pyridin-2-ylamine (Compound I) and an mTOR inhibitor selected from the group consisting of RAD rapamycin (sirolimus) and derivatives/analogs thereof such as everolimus or RAD001; CCI-779. ABT578, SAR543, ascomycin (an ethyl analog of FK506), AP23573, AP23841, KU-0063794. INK-128, EX2044, EX3855, EX7518, AZD08055 and OSI027, wherein the active ingredients are present in each case in free form or in the form of a pharmaceutically acceptable salt, and optionally at least one pharmaceutically acceptable carrier, for simultaneous, separate or sequential use for the treatment of mammalian target of rapamycin (mTOR) kinase dependent diseases.

The present invention provides a method to reduce or block the phosphorylation and activation of AKT by mTOR inhibitors composing administering a compound of formula (II) to a warm-blooded animal in need thereof. In another embodiment, the present invention provides a method of treating a proliferative disease dependent on acquired phosphorylation and activation of AKT during treatment with an mTOR inhibitor comprising administering a therapeutically effective amount of a compound of formula (I) to a warm-blooded animal in need thereof.

In another embodiment, the present invention relates to a method of treating a proliferative disease which has become resistant or has a decreased sensitivity to the treatment with an mTOR inhibitor comprising administering a therapeutically effective amount of a compound of formula (I) to a warm-blooded animal in need thereof. The resistance is e.g. due to posphorylation and activation of AKT.

In a further aspect the present invention provides a method for improving efficacy of the treatment of a proliferative disease with an mTOR inhibitor comprising administering a combination comprising a compound of formula (I) or 5-(2,6-di-morpholin-4-yl-pyrimidin-4-yl)-4-trifluoromethyl-pyridin-2-ylamine and a mTOR inhibitor to a warm-blooded animal in need thereof.

The mTOR inhibitor used according to the present invention may be selected from RAD rapamycin (sirolimus) and derivatives/analogs thereof such as everolimus or RAD001; CCI-779, ABT578, SAR543, ascomycin (an ethyl analog of FK506), AP23573, AP23841, KU-0063794, INK-128, EX2044, EX3855, EX7518, AZD08055 and OSI027. Particularly preferred mTOR inhibitors in accordance with the present invention are sirolimus and/or everolimus.

The term “mTOR kinase dependent diseases” includes but is not restricted to the following symptoms:
Organ or tissue transplant rejection, e.g. for the treatment of recipients of e.g. heart, lung, combined heart-lung, liver, kidney, pancreatic, skin or corneal transplants; graft-versus-host disease, such as following bone marrow transplantation;
Restenosis
Hamartoma syndromes, such as tuberous sclerosis or Coowden Disease
Lymphangiolelomyomatosis
Retinitis pigmentosis
Autoimmune diseases including encephalomyelitis, insulin-dependent diabetes mellitus, lupus, dermatomyositis, arthritis and rheumatic diseases
Steroid-resistant acute Lymphoblastic Leukaemia
Fibrotic diseases including scleroderma, pulmonary fibrosis, renal fibrosis, cystic fibrosis
Pulmonary hypertension
Immunomodulation
Multiple sclerosis
VHL syndrome
Carney complex
Familial adenonamtous polyposis
Juvenile polyposis syndrome
Birt-Hogg-Duke syndrome
Familial hypertrophic cardiomyopathy
Wolf-Parkinson-White syndrome
Neurodegenerative disorders such as Parkinson's, Huntington's, Alzheimer's and dementias caused by tau mutations, spinocerebellar ataxia type 3, motor neuron disease caused by SOD1 mutations, neuronal ceroid lipofucinoses/Batten disease (pediatric neurodegeneration)
wet and dry macular degeneration
muscle wasting (atrophy, cachexia) and myopathies such as Danon's disease,
bacterial and viral infections including M. tuberculosis, group A streptococcus, HSV type I, HIV infection
Neurofibromatosis including Neurofibromatosis type 1,
Peutz-Jeghers syndrome

Furthermore, “mTOR kinase dependent diseases” include cancers and other related malignancies. A non-limiting list of the cancers associated with pathological mTOR signaling cascades includes breast cancer, renal cell carcinoma, gastric tumors, neuroendocrine tumors, lymphomas and prostate cancer.

Examples for a proliferative disease are for instance benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, lymphomas, a mammary carcinoma or a leukemia.

The pharmaceutical compositions or combination in accordance with the present invention can be tested in clinical studies. Suitable clinical studies may be, for example, open label, dose escalation studies in patients with proliferative diseases. Such studies prove in particular the synergism of the active ingredients of the combination of the invention. The beneficial effects on proliferative diseases may be determined directly through the results of these studies which are known as such to a person skilled in the art. Such studies may be, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) may be administered in a fixed dose and the dose of agent (b) may be escalated. Each patient may receive doses of the agent (a) either daily or intermittent. The efficacy of the treatment may be determined in such studies, e.g., after 12, 18 or 24 weeks by evaluation of symptom scores every 6 weeks.

The administration of a pharmaceutical combination of the Invention may result not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically active ingredients used in the combination of the invention.

A further benefit may be that lower doses of the active ingredients of the combination of the invention may be used, for example, that the dosages need not only often be smaller but may also be applied less frequently, which may diminish the incidence or severity of side-effects. This is in accordance with the desires and requirements of the patients to be treated.

It is one objective of this invention to provide a pharmaceutical composition comprising a quantity, which may be jointly therapeutically effective at targeting or preventing proliferative diseases a combination of the Invention. In this composition, agent (a) and agent (b) may be administered together, one after the other or separately in one combined unit dosage form or in two separate unit dosage forms. The unit dosage form may also be a fixed combination.

The pharmaceutical compositions for separate administration of agent (a) and agent (b) or for the administration in a fixed combination, i.e. a single galenical composition comprising at least two combination partners (a) and (b), according to the invention may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers or diluents, especially suitable for enteral or parenteral application.

Suitable pharmaceutical compositions may contain, for example, from about 0.1% to about 99.9%, preferably from about 1% to about 60%, of the active ingredient(s). Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise (i) administration of the first agent (a) in free or pharmaceutically acceptable salt form and (ii) administration of an agent (b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

The following examples are illustrative only and not intended to be limiting.

### EXAMPLE 1

### Effect of the Combination of RAD001 (Everolimus) with Compound I in BT474 and MDA-MB-231 Breast Tumor Cells

### Material Methods

### 1. Preparation of Compounds

The compound RAD001 is synthesized by Novartis Pharma AG. A 20 mM stock solution is prepared in DMSO and stored −20° C. A 10 mM stock solution of the Compound I is prepared in DMSO and stored at −20° C.

### 2. Cells and Cell Culture Conditions

Human breast carcinoma BT474 (ATCC HTB-26) and MDA-MB-231 (ATCC HTB-20) are obtained from the American Type Culture Collection (ATCC, Rockville, Md. USA). BT474 cells are maintained in Hybri-Care medium (ATCC) supplemented with 10% v/v fetal calf serum and 2 mM L-glutamine. MDA-MB-231 cells are grown in RPMI 1640 medium (Amimed, Allschwil, Switzerland) supplemented with 10% v/v fetal calf serum and 2 mM L-glutamine. All media are supplemented with 100 μg/mL penicillin/streptomycin and cells are maintained at 37° C. In 5% CO2.

### 3. Cell Treatment and Cell Extraction

BT474 and MDA-MB-231 cells are seeded at a density of 3.3×10⁴ cells/cm² and 1.6×10⁴ cells/cm², respectively, and incubated for 48 h at 37*C and 5% CO₂, prior to treatment with DMSO vehicle, 20 nM RAD001 and/or various concentrations of Compound I for 24 h. Cell lysates are prepared as follows. Culture plates are washed once with ice-cold PBS containing 1 mM PMSF and once with ice-cold extraction buffer [50 mM Hepes (pH 7.4), 150 mM NaCl, 25 mM β-glycerophosphate, 25 mM NaF, 5 mM EGTA, 1 mM EDTA, 15 mM PPi, 2 mM sodium orthovanadate, 10 mM sodium molybdate, leupeptin (10 μg/mL), aprotinin (10 μg/mL), 1 mM DTT and 1 mM PMSF]. Protease inhibitors are purchased from SIGMA Chemical. St. Louis. Mo. Cells are extracted in the same buffer, containing 1% NP-40 (SIGMA Chemicals). The extracts re homogenized, cleared by centrifugation, aliquoted and frozen at −80° C. Protein concentration are determined with the BCA Protein Assay (Pierce. Rockford, Ill. USA).

### 4. Immunoblotting

Twenty micrograms of cell extracts are resolved electrophoretically on 12% denaturing sodium dodecyl sulfate polyacrylamide gels (SDS-PAGE) and transferred to polyvinylidene difluoride filters (PVDF; Millipore Corporation, Bedford, Mass., USA) by wet-blotting (1 h at 250 mA) and probed overnight at 4° C. with the following primary antibodies:

anti-phospho-Akt (Ser473) (clone 14-05; 1:2000) obtained from DAKO (Glostrup, Denmark) and diluted in PBS, 0.5% v/v Tween.
anti-phospho-Akt (T308) (cat #9275; 1:1000) obtained from Cell Signaling Technology (Beverly, Mass., USA) and diluted in PBS, 0.1% v/v Tween.
anti-Akt (cat #1085-1; 1:5000) obtained from Epitomics (Burlingame, Calif., USA) and diluted in PBS, 0.5% v/v Tween.

Anti-Actin (cat #MAB1501; 1:20,000) obtained from Chemicon (Billerica. Mass. USA) and diluted in PBS, 0.1% v/v Tween.

After incubation with the appropriate primary antibody (above), decorated proteins are revealed using horseradish peroxidase-conjugated anti-mouse or anti-rabbit immunoglobulins followed by enhanced chemiluminescence (ECL Plus kit; Amersham Pharmacia Biotech, Buckinghamshire, UK) and quantified using Quantity One Software (Bio-Rad, Munich. Germany).

1. A pharmaceutical combination comprising
a) a compound of formula (I) 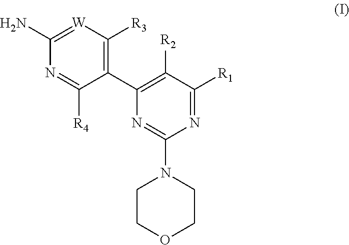
or a stereoisomer, tautomer, or pharmaceutically acceptable salt thereof, wherein,
W is CR_{w} or N, wherein R_{w} is selected from the group consisting of
(1) hydrogen,
(2) cyano,
(3) halogen,
(4) methyl,
(5) trifluoromethyl,
(6) sulfonamido;
R₁ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NR₁ₐSO₂R_{1b},
(18) —OCOR₁ₐ,
(19) —OR₁ₐ,
(20) —SR₁ₐ,
(21) —SOR₁ₐ,
(22) —SO₂R₁ₐ, and
(23) —SO₂NR₁ₐR_{1b},
wherein R₁ₐ, and R_{1b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
R₂ is selected from the group consisting
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b},
wherein R₂ₐ, and R_{2b} are independently selected from the group consisting of
(a) hydrogen, and
(b) substituted or unsubstituted alkyl;
R₃ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₃ₐ,
(13) —NR₃ₐR_{3b},
(14) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ, and
(20) —SO₂NR₃ₐR_{3b},
wherein R₃ₐ, and R_{3b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl; and
R₄ is selected from the group consisting of
(1) hydrogen, and
(2) halogen
and
b) at least one mTOR inhibitor.

2. The pharmaceutical combination of claim 1 wherein the compound of formula (I) is 5-(2,6-di-morpholin-4-yl-pyrimidin-4-yl)-4-trifluoromethyl-pyridin-2-ylamine.

3. A pharmaceutical combination according to claim 1 wherein the mTOR inhibitor is selected from RAD rapamycin (sirolimus) and derivatives/analogs thereof such as everolimus or RAD001; CCI-779, ABT578, SAR543, ascomycin (an ethyl analog of FK506), AP23573, AP23841, KU-0063794, INK-128, EX2044, EX3855, EX7518, or compounds that bind to the ATP-binding cleft of mTOR, such as AZD08055 and OSI027

4. A pharmaceutical combination according to claim 1 for the treatment and prevention of a mammalian target of rapamycin (mTOR) kinase dependent diseases.

5. A method of treating or preventing a mammalian target of rapamycin (mTOR) kinase dependent diseases by administering the pharmaceutical combination of claim 1 to a warm blooded animal in need thereof.

6. A combination of a compound of formula (I) 5-(2,6-di-morpholin-4-yl-pyrimidin-4-yl)-4-trifluoromethyl-pyridin-2-ylamine (Compound I) and an mTOR inhibitor selected from the group consisting of RAD rapamycin (sirolimus) and derivatives/analogs thereof such as everolimus or RAD001, CCI-779, ABT578, SAR543, ascomycin (an ethyl analog of FK506), AP23573, AP23841, KU-0063794, INK-128, EX2044, EX3855, EX7518, or compounds that bind to the ATP-binding cleft of mTOR, such as AZD08055 and OSI027, wherein the active ingredients are present in each case in free form or in the form of a pharmaceutically acceptable salt, and optionally at least one pharmaceutically acceptable carrier, for simultaneous, separate or sequential use for the treatment of breast cancer, renal cell carcinoma, gastric tumors, neuroendocrine tumors, lymphomas, prostate cancer, treatment of organ or tissue transplant rejection; restenosis, tuberous sclerosis, Cowden Disease, lymphangioleiomyomatosis, retinitis pigmentosis, an autoimmune diseases, steroid-resistant acute Lymphoblastic Leukaemia, a fibrotic diseases, pulmonary hypertension, Immunomodulation; multiple sclerosis; VHL syndrome; Carney complex; familial adenonamtous polyposis; juvenile polyposis syndrome; Birt-Hogg-Duke syndrome; familial hypertrophic cardiomyopathy; Wolf-Parkinson-White syndrome; a Neurodegenerative disorder; wet and dry macular degeneration; muscle wasting (atrophy, cachexia) or a myopathies; a bacterial or viral infection; Neurofibromatosis or Peutz-Jeghers syndrome.

7. A method of treating a proliferative disease dependent on acquired phosphorylation and activation of AKT in the treatment with an mTOR inhibitor comprising administering a therapeutically effective amount of a compound of formula (I) to a warm-blooded animal in need thereof.

8. The method according to claim 7, wherein the disease to be treated is breast cancer, renal cell carcinoma, gastric tumors, neuroendocrine tumors, lymphomas and prostate cancer.

9. The method according to claim 7, wherein the disease to be treated is organ or tissue transplant rejection; restenosis, tuberous sclerosis, Cowden Disease, lymphangioleiomyomatosis, retinitis pigmentosis, an autoimmune diseases, steroid-resistant acute Lymphoblastic Leukaemia, a fibrotic diseases, pulmonary hypertension, Immunomodulation; multiple sclerosis; VHL syndrome; Carney complex; familial adenonamtous polyposis; juvenile polyposis syndrome; Birt-Hogg-Duke syndrome; familial hypertrophic cardiomyopathy; Wolf-Parkinson-White syndrome; a Neurodegenarative disorder; wet and dry macular degeneration; muscle wasting (atrophy, cachexia) or a myopathies; a bacterial or viral infection; Neurofibromatosis or Peutz-Jeghers syndrome.

10. A method of treating a proliferative disease which has become resistant or has a decreased sensitivity to the treatment with an mTOR inhibitor comprising administering a therapeutically effective amount of a compound of formula (I) to a warm-blooded animal in need thereof.

11. A method for improving efficacy of the treatment of a mammalian target of rapamycin (mTOR) kinase dependent disease with an mTOR inhibitor comprising administering a combination comprising 5-(2,6-di-morpholin-4-yl-pyrimidin-4-yl)-4-trifluoromethyl-pyridin-2-ylamine and a mTOR inhibitor to a warm-blooded animal in need thereof.
